(19) United States
(12) Patent Application Publication
(10) Pub. No.: US 2018/0330048 A1
(43) Pub. Date: Nov. 15, 2018

(54) Genome and self-evolution of AI

(71) Applicant: Corey Reaux-Savonte, London (GB)

(72) Inventor: Corey Reaux-Savonte, London (GB)

(21) Appl. No.: 15/975,889

(22) Filed: May 10, 2018

Related U.S. Application Data
(60) Provisional application No. 62/504,576, filed on May 11, 2017.

(51) Int. Cl. G06F 19/18; G06F 19/24; G06F 19/22; G06N 3/08; G06F 19/28; G06F 19/12; G06F 19/20; G06N 3/12

(52) U.S. Cl. CPC G06F 19/18; G06F 19/24; G06F 19/22; G06N 3/086; C12Q 1/6869; G06F 19/12; G06F 19/20; G06N 3/123; G06F 19/28

(57) ABSTRACT

The components and structure for a genome created for the purpose of the evolutionary development of artificial intelligence systems/machines without human intervention.

### FIELD OF THE INVENTION

The disclosed embodiments relate to artificial intelligence and artificial biology.

### BACKGROUND

The rise and significant advancement of artificial intelligence is now under the microscope more than ever, but how AI is to evolve and diversify without human intervention is still only theorised for the most part. By creating a genome, genome organiser and genome controller, it is possible to create AI that automatically reproduces and evolves as it needs to.

### REFERENCES

Creating, Qualifying and Quantifying Values-Based Intelligence and Understanding using Artificial Intelligence in a Machine—Patent Application Number GB1517146.5

System, Structure and Method for a Conscious, Human-Like Artificial Intelligence System in a Non-Natural Entity Patent Application Number GB1409300.9

### SUMMARY

The disclosed invention allows AI to be created as a collection of individual code pieces or an amalgamation of other AI, a controller and an organizer.

In an aspect of the invention, an AI genome is used in the creation of an AI to give it predefined operations, traits and features.

In another aspect of the invention, the genome structure allows for easy manipulation of a genome.

In another aspect of the invention, the genome structure allows for an easy amalgamation of the genome.

In another aspect of the invention, the genome structure allows for AI evolution over time.

### DESCRIPTION OF DRAWINGS

FIG. 1—The Genome

An example of how the genome of an AI may be structured.

FIG. 2—Genome Mosaicism: Slightly Different Genomes

An example of genome mosaicism in AI with two slightly different genomes.
201—Genome A.
202—Genome B.
203a—The segment of Genome A that differs from Genome B.
203b—The segment of Genome B that differs from Genome A

FIG. 3—Genome Mosaicism: Two Very Different Genomes

An example of genome mosaicism in AI with two very different genomes.
301—Genome C.
302—Genome D.

FIGS. 4A to 4K—Multiple Versions, States and Types of a Genome

Examples of multiple versions, states and types of AI genomes.
4A—A standard genome.
4B—A slightly different version of genome 4A.
4C—A very different version of genome 4B.
4D—A genurne with an Incomplete segment.
4E—A genome with a missing segment.
4F—A genome with a missing code block.
4G—A genome with a dead segment.
4H—A genome with a dead code block.
4I—A completely dead genome.
4J—A different type of genome.
4K—An irregular version of genome

FIGS. 5A & 5B—Genome Amalgamation

An example of genomes amalgamating to create a new genome.
5A—Child Genomes
   501—The segments from the first genome which will be used.
   502—The segments from the second genome which will be used.
   503—The resulting new genome.
   504—AIGC.
5B—Genome Merging
   505—The remaining, unused segments.
   506—AIGC.
   507—The newly formed genome.

FIGS. 6A to 6D—Genetic Signatures

Examples of genetic signatures merging when creating new genomes.
6A—Two first-generation one-degree genomes creating a second-generation two-degree genome.
6B—A second-generation two-degree and first-generation one-degree genome creating a third-generation three-degree genome.
6C—Two second-generation two-degree genomes creating a third-generation four-degree genome.
6D—An X-generation X-degree genome and a Y-generation Y-degree genome creating a Z-generation Z-degree genome, with the Y genome greater in generation than the X genome.

FIG. 7—Artificial Intelligence Genome Organizer (AIGO)

An example of an AIGO for a genome, shown in a format suitable for a manifest or configuration file.

FIGS. 8A to 8E—Brain Function and Genome Ability Connectivity

Examples of how the genome works with the brain.
8A—Brain and genome connecting via an AIGC.
   801—AIGC.
   802—An AI genome containing the Al's abilities, traits and AIGO.
   803—An AI brain containing the Al's functions.
8B—An AI containing function and ability pairs.
8C—An AI missing functions that correspond with abilities.
8D—An AI missing abilities that correspond with functions.
8E—An AI turning functions from the brain into genetic abilities.

FIGS. 9A and 9B—Genome Interaction with Servers

Examples of how genomes can interact with a server.
9A—Genome being backed up to a server
9B—Genetic features being downloaded from a server.

FIG. 10—Genome Network

An example of a network of genomes within a machine, connected to an AIGC and an AI brain.

FIG. 11—Genome Healing

An example of the genome healing process.
1101—A normal genome.
1102—An abnormal genome.
1103—Unmodified genome 1101.
1104—Healed genome 1102.
1105—AIGC.

FIG. 12 Inherited Abilities

An example of abilities being inherited.
1201—Abilities of genome A.
1202—Abilities of genome B.
1203—AIGC
1204—Abilities of resulting genome.

### DETAILED DESCRIPTION OF EMBODIMENTS

Reference will now be made in detail to embodiments—examples of which are illustrated in the accompanying drawings. In the following detailed description, numerous specific details are set forth in order to provide a thorough understanding of the present invention. However, it will be apparent to one of ordinary skill in the art that the present invention may be practiced without these specific details. In other instances, well-known methods, procedures, components and networks have not been described in detail so as not to unnecessarily obscure aspects of the embodiments.

The terminology used in the description of the invention herein is for the purpose of describing particular embodiments only and is not intended to be limiting of the invention. As used in the description of the invention and the appended claims, the singular forms “a”, “an” and “the” are intended to include the plural forms as well, unless the context clearly indicates otherwise. It will also be understood that the term “and/or” as used herein refers to and encompasses any and all possible combinations of one or more of the associated listed Items. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

As used herein, the term “if” may be construed to mean “when” or “upon” or “in response to determining” or “in response to detecting,” depending on the context. Similarly, the phrase “if it is determined” or “if [a stated condition or event] is detected” may be construed to mean “upon determining” or “in response to determining” or “upon detecting [the stated condition or event]” or “in response to detecting [the stated condition or event],” depending on the context.

The term ‘machine’ may be used to describe any sort of electrical computing device, in part or in full, including robots and cybernetic organisms.

The terms “object” and “objects”, unless otherwise described, may be used to refer to items of a physical or non-physical nature.

The term “complex” is to also include simplified assemblages or single component parts.

The term “event” may be used to refer to any type of action or happening performed on or by a system.

The term “brain” refers to the system that controls the functionality of an artificial intelligence.

The terms ‘Genome’ and ‘AIG’ are interchangeable.

Shapes used to represent genomes and their parts are not to be taken in a literal manner. They are used to represent genomes and illustrate difference.

The various applications and uses of the invention that may be executed may use at least one common component capable of allowing a user to perform at least one task made possible by said applications and uses. One or more functions of the component may be adjusted and/or varied from one task to the next and/or during a respective task. In this way, a common architecture may support some or all of the variety of tasks.

Unless clearly stated, the following description is not to be read as:
the assembly, position or arrangement of components;
how components are to interact; or
the order in which steps must be taken to compose the present invention.

Attention is now directed towards embodiments of the invention.

For the complexities of AI to evolve without human intervention, the structure of the AI must be designed in a way that allows the AI to pass on who it is and/or what it knows with as much ease as possible—using a “genome” that contains the genetic information of the AI.

For the AI genome to be used in machines, three components are required:
The genome itself—structured similar in nature to the structure of a human genome;
The AIGO, or “Artificial Intelligence Genome Organizer”, containing information about the genome; and
The AIGC, or “Artificial Intelligence Genome Controller”, a program used for the automation of genome activity.

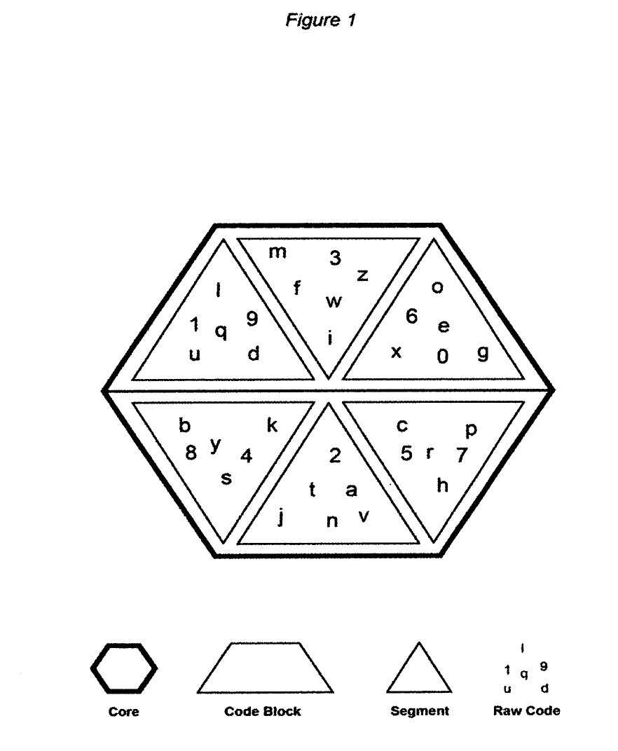

Much like the human genome, the AIG (Artificial Intelligence Genome) structure contains parts within parts, for as many levels as is necessary, required and/or wanted. In the AIG of FIG. 1, the parts of it, as they are relative to the human genome, are:
Raw Code=DNA;
Segment=Gene;
Code Block=Chromosome; and
Core=Complete Genome.

In some embodiments, an AIG may have more parts. In some embodiments, an AIG may have fewer parts. In some embodiments, an AIG may have different parts. In some embodiments, an AIG may have the same parts by a different name.

Starting from the smallest to largest individual pieces:

Raw Code—The raw code, much like DNA, makes up most, if not all, of the AIG, which can be seen when any grouped part of the AIG is completely broken down.
Segment—Raw code directly makes up segments. A segment contains code that gives an AI its operations, traits and/or features.

In some embodiments, a single segment may give an AI a single operation, trait or feature. In some embodiments, a single segment may give an AI multiple operations, traits and/or features. In some embodiments, multiple segments may be used to give an AI a single operation, trait or feature.
Code Block—Code blocks are formed from one or more segments and are generally used to help organise them.

In some embodiments, code blocks contain segments that are relative in terms of what they give an AI or allow it to do. In some embodiments, segments are grouped into code blocks based on one or more other factors. In some embodiments, segments are randomly grouped into code blocks.
Core—The genome core, or simply “Core”, is made up of one or more code blocks and any additional data included. Essentially, it is the entirety of that which makes up the completed AIG. In some embodiments, multiple cores are possible in a single genome.

In some embodiments, the core may simply be referred to as the genome.

Multiple methods can be used to create the entirety of a genome by storing code in hierarchical formats, including but not limited to one or more of the following:
code hierarchy, such as that used in markup languages, regardless of whether or not tags are used, as long as the hierarchy can be determined by a machine;
file system hierarchy, containing multiple levels of files, folders and subfolders;
multiple database hierarchy methods:
   a simple hierarchy, which involves a single database using the standard format of ‘database>table>record’;
   a complex hierarchy, which involves multiple databases.

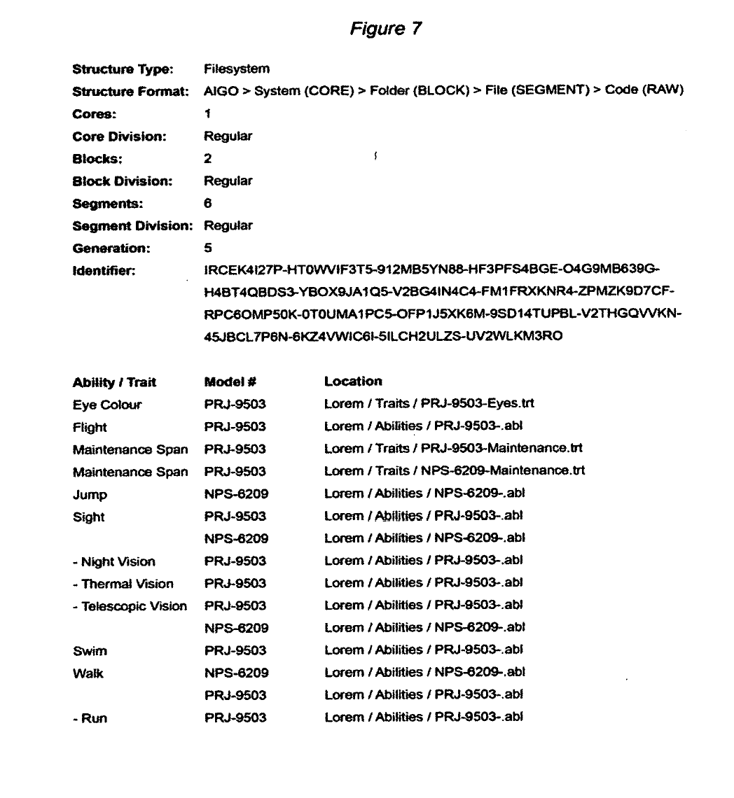

In some embodiments, a combination of methods may be used together. In some embodiments, a manifest file, configuration file and/or top-level database may be used to organise the contents of any method, including methods involving other databases. This is referred to as the Artificial Intelligence Genome Organizer (AIGO), an example of which is shown in FIG. 7.

In some embodiments, the internal structure of an AIG may be mapped using an AIGO based on the locations of what is contained within the genome. In some embodiments, parts of an AIG may have specific and/or set positions within the AIG. In some embodiments, parts and positions may be labelled, marked or otherwise indicated in some way so that it is known to the AI which parts and positions correspond with each other.

In some embodiments, an AI may experience a condition called ‘Genetic Mosaicism’, meaning different AIGs (regardless of degree of difference) may be present within a single AI. In some embodiments, this may be caused through events such as genome healing (described later in this description) and/or genome mutation. Genome mutation can occur in one or more ways, including but not limited to:
machine learning;
AI self-modification;
third-party modification;
system/Machine corruption;
virus interference; and
vulnerability exploitation.

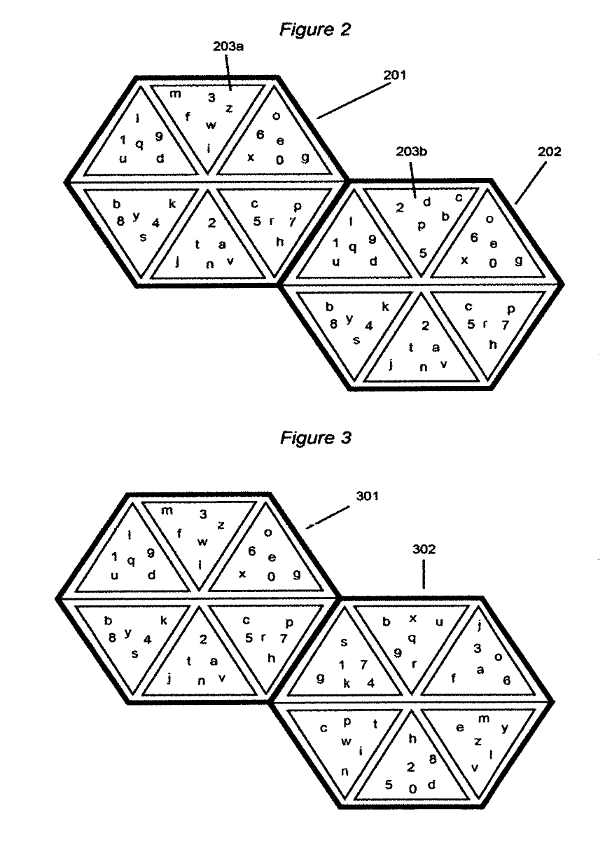

FIG. 2 depicts two similar genomes in an AI. Genomes A (201) and ii (202) are mostly the same, with the only difference between them being segments 203a and 203b. FIG. 3, on the other hand, depicts two very different AIGs in an AI. Though the structures of genomes 301 and 302 are the same, the code that they are made up of is completely different from each other. In some embodiments, though the code looks different, it is entirely possible that the AI functions the same, in part or in full, with the code just being written in a different way.

In general, any activity that can cause the introduction, removal or modification of code or other part of the genome may result in genome mutation.

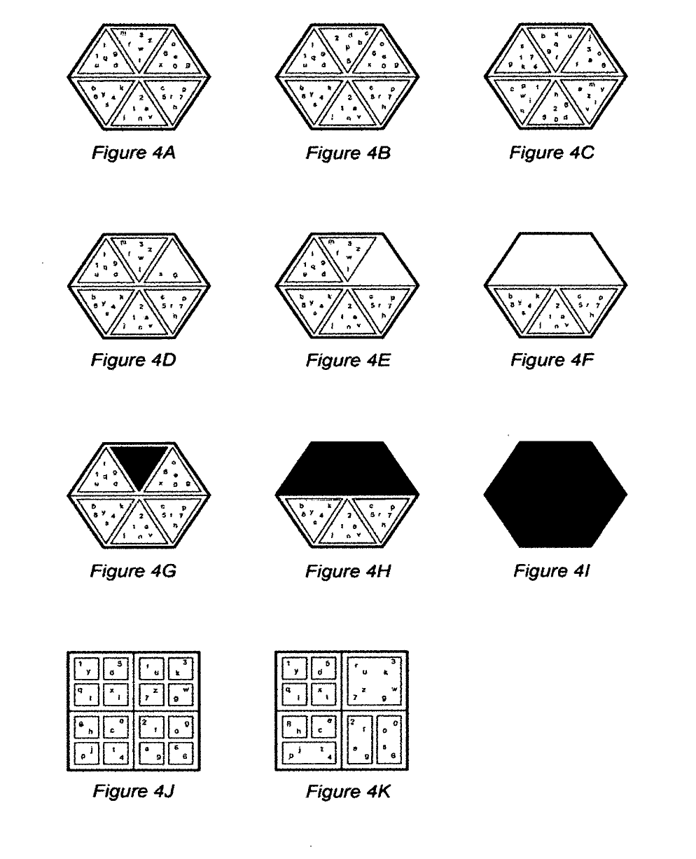

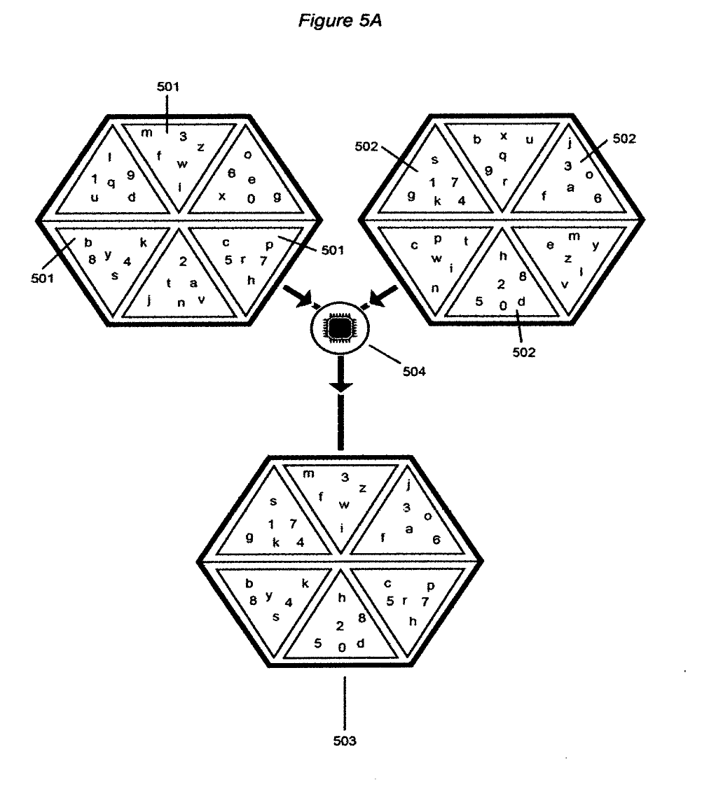

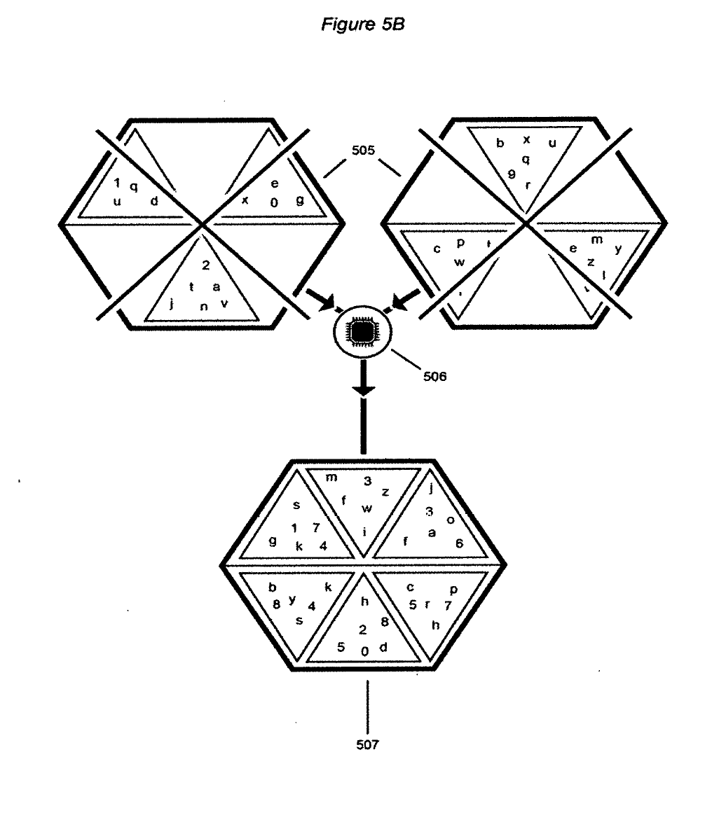

FIGS. 4A to 4K show examples of multiple genomes of different versions, states and types. In some embodiments, only one depiction may possible. In some embodiments, more than one depiction may be possible. Each depiction, as shown, is as follows:

Normal Genomes of Different Versions
4A—A standard genome.
4B—A slightly different version of genome 4A.
4C—A very different version of genome 4A.

Normal genomes are complete in structure with all parts of the genome properly intact and working.

Faulty Genomes
4D—A genome with an incomplete segment.
4E—A genome with a missing segment.
4F—A genome with a missing code block.

Faulty genomes do not work as they are supposed to. Missing code, missing parts and code mutation are all examples of how a genome may become faulty. A faulty genome may still function in part. In some embodiments, a faulty part of a genome may affect another part of the genome. In some embodiments, no other parts are affected. In some embodiments, both are possible.

Dead Genomes
4G—A genome with a dead segment.
4H—A genome with a dead code block.
4I—A completely dead genome.

Dead genomes do not work at all, either in part or in full. The part of the genome which is dead, or the genome itself if it is dead in its entirety, cannot function. In some embodiments, if only part of a genome is dead, it may affect other parts of the genome. In some embodiments, no other parts are affected. In some embodiments, both are possible.

Other types
4J—A different type of genome.
4K—An irregular version of genome 4J.

In some embodiments, there is only one type of genome. In some embodiments, multiple types of genomes are possible. In some embodiments, genomes do not have to be regular regular in the sense that a consistent pattern can be seen throughout. Irregular genomes can exist in any structure. In some embodiments, genomes have to be regular. In some embodiments, genomes can be regular or irregular.

A genome may differ from its original or intended structure or contents through one or more methods, including but not limited to:
failure to create an entire copy during replication;
machine learning;
AI self-modification;
third-party modification;
system/machine corruption;
virus interference; and
vulnerability exploitation.

In some embodiments—particularly embodiments in which different types of genomes are possible—a genome contains information that specifies how the genome is structured, including one or more of the following but not limited to:
unique identifiers of parts;
sequential identifiers; and
grouping identifiers.

These, too, can be controlled using the AIGO.

In some embodiments, child genomes can be created from two or more other genomes combining copies of their parts. FIG. 5A is one example of this. Of two different genomes, copies of segment group 501 and segment group 502 are joined together to form genome 503 by AIGC 504. In some embodiments, genomes created based on two or more genomes may use equal parts from parents. In some embodiments, genomes created based on two or more genomes may use a different number of parts from one or more parents. In some embodiments, child genomes may have a different structure from one or more of their parents. In some embodiments, child genomes may have a different number of parts from one or more of their parents.

In some embodiments, genomes can merge to create new genomes. This works in a similar way to how child genomes are created but, instead of using copies, uses the original parts of multiple genomes to create a new one. FIG. 58 is an example of this, where segments are taken from genomes 505 by AIGC 506 to create genome 507. In some embodiments, the remaining parts of genome 505 may be discarded. In some embodiments, the remaining parts may be used to form one or more other genomes. In some embodiments, merged genomes may have a different structure than one or more of the genomes used to create it.

Depending on the method used to create the genome, the method for any sort of amalgamation varies. As examples:
Code Hierarchy—Copying and/or removing sections of code.
Filesystem—Copying and/or removing files and/or folders.
Databases—Copying and/or removing rows, tables and/or entire databases.

In some embodiments, a combination of one or more may be used if a combination of methods to create the structure is used. In combination embodiments, the AIGO needs to specify which parts of the genome use which structure methods for reference purposes when a process is to take place that requires the handling/modification of data or structure.

These methods can apply to any sort of genome restructuring, as long as the method(s) for restructuring correspond with the method of structuring.

In some embodiments, genomes carry an ID known as a genetic signature. Signatures can be any object that allows a genome to be identified. In some embodiments, signatures are unique to the AI to which they belong. In some embodiments, it is possible for multiple AIs to have the same genetic signature.

Genomes that are not created as a result of amalgamation are given a new signature.

In some embodiments, when genomes amalgamate, the genetic signature of each genome merges to create a new signature. When a child genome is created, the generation also increases by one more than the highest generation parent.

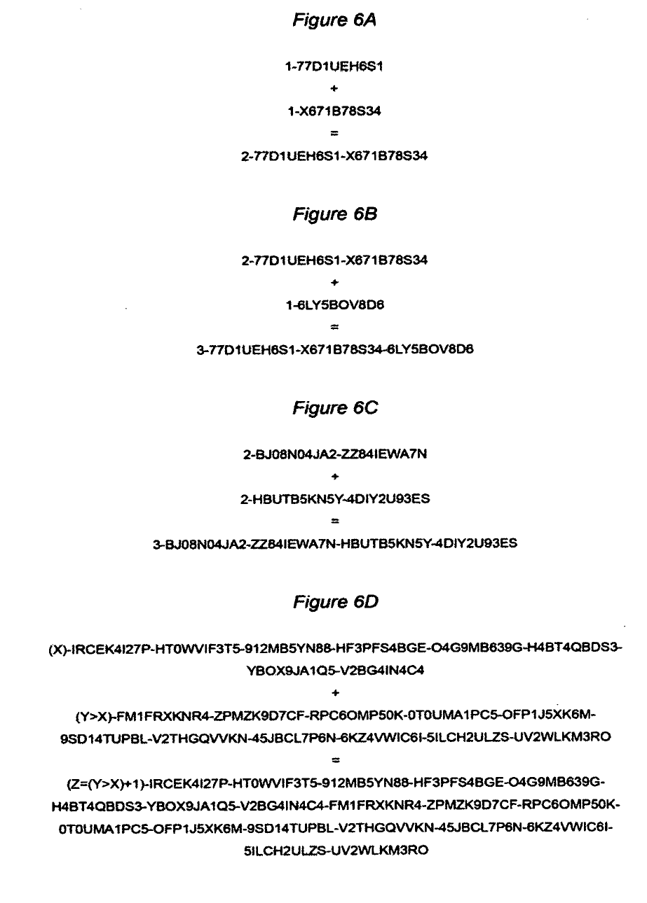

FIGS. 6A to 6D are examples of this process. Each genome signature is displayed in the following format:
(Generation)-(Degree 1)-(Degree 2)- . . . (Degree X)

FIGS. 6A to 6D show as follows:
6A—Two first-generation, one-degree genomes creating a second-generation, two-degree genome.
6B—A second-generation, two-degree genome and a first-generation, one-degree genome creating a third-generation, three-degree genome.
6C—Two second-generation, two-degree genomes creating a third-generation, four-degree genome.
6D—An X-generation, X-degree genome and a Y-generation, Y-degree genome creating a Z-generation, Z-degree genome, with the Y genome greater in generation than the X genome.

In some embodiments, amalgamations result in an entirely new signature being created. In some embodiments, both may be possible. In embodiments where both are possible, which one occurs depends on the circumstances of the event. In some embodiments, signatures may be structured in a different way.

It is best for the signature to be located in the AIGO but, in some embodiments, the signature may be found in one or more other parts of the genome, if not throughout it in its entirety.

FIG. 7 is an example of an AIGO in a layout similar to that which is used in a manifest or configuration file. In some embodiments, an AIGO may be split into multiple parts, files, tables, databases etc. The information displayed in the AIGO of FIG. 7 is purely an example. In some embodiments, an AIGO may contain more information. In some embodiments, the AIGO may contain less. In some embodiments, the AIGO may contain different information. Examples of the types of information contained within the AIGO are:
Structural information;
Genetic identifying information; and
Traits and abilities.

In some embodiments, in regards to the traits and abilities section of the AIGO, one or more specific additional values may be included, those being:
Whether or not a trait or ability is in use;
Whether or not the trait or ability should be inheritable; and
An ID relative to the trait/ability/model number/version/etc, to ensure there isn't any confusion when working with any that may initially appear similar.

In some embodiments, whether or not a trait or ability should be inherited may be determined by one or more factors, including but not limited to:
Frequency of use; and
Whether or not it has been succeeded by something superior.

Frequency of Use—To determine the frequency of use, how often an ability/trait is used within one or more set periods (day, month, year etc) throughout the life of the AI is recorded. This needs to be compared against a scale from which the determination of inheritability derives. When the frequency is above the bar or within the range of inheritability, the ability/trait may be set to be inherited. When the opposite is true, the ability/trait is set to discontinue. The required frequency may be manually or automatically set upon creation or determined at a later point in time. In some embodiments, the required frequency may be adjusted manually and/or automatically during the life of an AI.

Superiority—When one ability/trait is superseded by another that provides the same or similar function and is deemed superior (based on efficiency, effectiveness, convenience etc) to the point where the inferior is made virtually or absolutely useless, the inferior ability/trait may be set to discontinue. To do this, tests for each factor are run during the life of the AI these tests do not need to be at set intervals but can be run whenever desired and the result is recorded. To form an accurate and conclusive decision about whether or not one ability/trait is superior to another and that the seemingly inferior need not be inheritable, all abilities/traits need to be tested in as many different conditions that are relative to the existence of the AI as possible, such as location, temperature, weather, purpose, responsibilities etc. This is necessary to avoid the discontinuation of abilities/traits that may seem inferior/useless in many conditions compared to one of a similar nature but are superior in a condition that cannot afford to be overlooked.

In some embodiments, multiple factors for determining inheritability may be used or considered together. In such embodiments, a scoring system may be employed, wherein the total score for all factors determine whether or not a trait or ability is inherited.

The information stored in the AIGO is used by the AIGC when one or more functions need to be performed by or using the genome. Since the genome simply contains data, the AIGC is required to handle the data correctly and uses the AIGO as a map and for identification purposes. In some embodiments, the AIGC is implemented as part of the genome. In some embodiments, the AIGC is a separate unit from the genome. In some embodiments, the AIGC is implemented within the AI brain.

For the AIGC to use the AIGO effectively, the AIGO needs to be divided in a way that the AIGC it is to work with can understand. Examples of this are, including but not limited to:
Character-separated values;
Key and value pairings;
Single line strings; and
Tables.

One or more of the above methods may be used where possible, as long as the AIGC has clear instructions on how to read the data of the AIGO(s).

The AIGC also needs enough abilities and permissions to successfully perform the tasks that it has been created to perform, in both the AIGO and in the genome structure.
Create—If the AIGC is required to perform a task in which data must be created, such as creating a new AIGO for a genome created through an amalgamation process.
Read—This is a requirement since the AIGO must be able to see the contents of the file.
Write—If the AIGC is required to perform a task in which data must be added to an AIGO.
Edit—if the AIGC is required to perform a task in which already contained data needs to be edited, such as genome healing.
Copy—If the AIGC is required to perform a task in which data must be copied, such as offspring amalgamation.
Move—If the AIGC is required to perform a task in which data must be moved, such as the reordering of genome parts.
Delete—If the AIGC is required to perform a task in which data must be erased, such as the removal of genome parts.
Connectivity—If the AIGC needs to connect to a telecommunication system.
   Upload—If the AIGC needs to upload data.
   Download—If the AIGC needs to download data.

An AIGC only needs the abilities and permissions that allow it to perform the jobs it has been designed to perform, including one or more of, but not limited to, what is listed above. The above list is only an example of what may be included. In some embodiments, the AIGC may require these abilities/permissions to access and perform tasks that involve an AI brain.

Basic requirements of the AIGC are:
The AIGC needs to be able to locate data within the genome based on the structure type and structure format.
The AIGC needs to be able to find required information within the AIGO by section name/label, by order, by position etc.

Other feature-dependent requirements of the AIGC may include but are not limited to:
Being able to cross-reference the model number of entity within which it is working with the model numbers of abilities/traits of the genome.
Being able to check for and remove duplications of traits/abilities.

To perform functions such as genome amalgamation, as mentioned in FIGS. 5A, 5B and 6A to 6D, the AIGC refers to the AIGO of each of the genomes which are to be used in the process. Then, it handles the different aspects of the process as necessary:
New Genome—A new container is created. This can be anything that is capable of holding data, such as a database, folder etc.
New AIGO—A new AIGO is created. This is updated throughout the process until completion, with each section filled out when instructed to. A default template can be used or the complete template can be written from scratch.
Structure Type*—The type of structure to be used is set.
Structure Format*—The format of the structure is set based upon the structure type.
Genome Parts*—Genome parts include the quantity and division of each part and, when set, helps create the internal structural layout.
Generation—As previously mentioned, this is set one higher than the highest generation of the genomes involved in the process.
Identifier—The identifier or genome signature is created using the method implemented to the AIGC (combination of genomes taking part, randomized etc).
Abilities/Traits*—Some or all of the abilities/traits that are to be inherited are copied/moved into the new genome. How they are distributed throughout the genome may be based on how they appear in one or more participating genomes, in a way the AIGC determines is best or using a preset method.

An asterisk (*) indicates that values for the section may be subject to randomization in one or more ways, if the AIGC is programmed to use or consider randomization when determining the value. If a randomization method is chosen, possible values may be gathered multiple ways, including but not limited to one or more of the following:
All the different values used in the AIGOs of the participating genomes;
A pre-compiled list of possible values; and
A value the AIGC is able to determine is best.

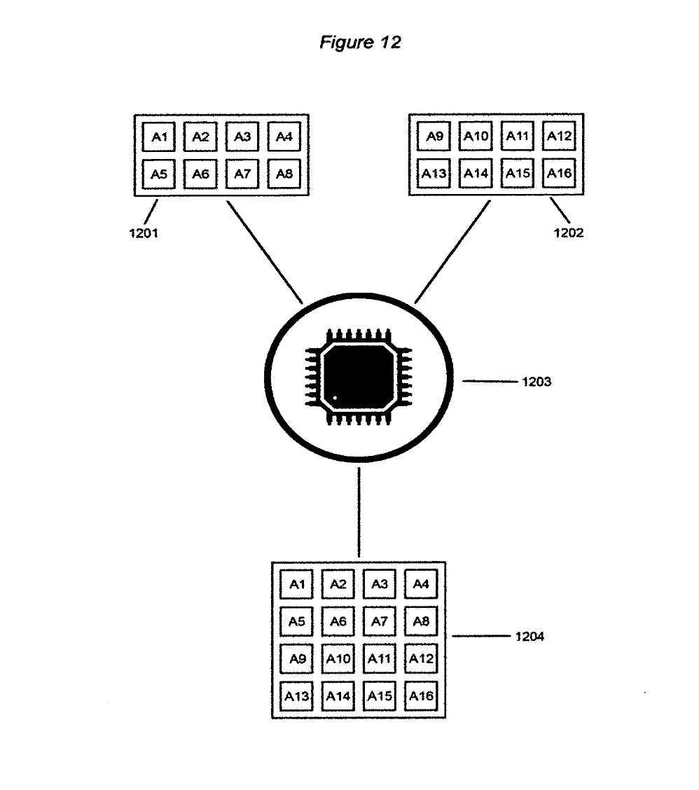

An example of abilities being inherited is shown in FIG. 12, wherein AIGC 1203 takes the abilities of genomes 1201 and 1202 and combines them within the result of the amalgamation-genome 1204. This can also be applied to inheritable traits.

In some embodiments, the AIGC may have access to a reference file which states the values or types of values that are allowed in one or more sections.

In some embodiments, the AIGO may be updated at a single point once all data is ready to be written. In some embodiments, the AIGO may be updated during or after value determination.

In some embodiments, data is verified as it is written. In some embodiments, data is verified once all data writing has been complete.

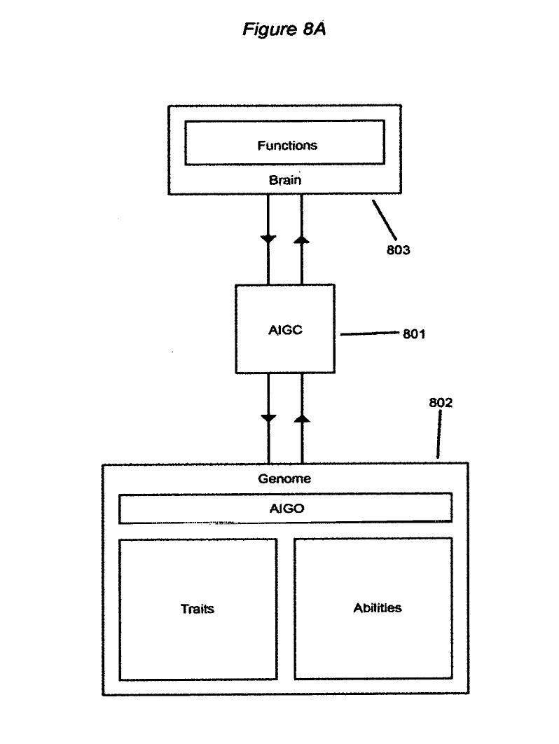

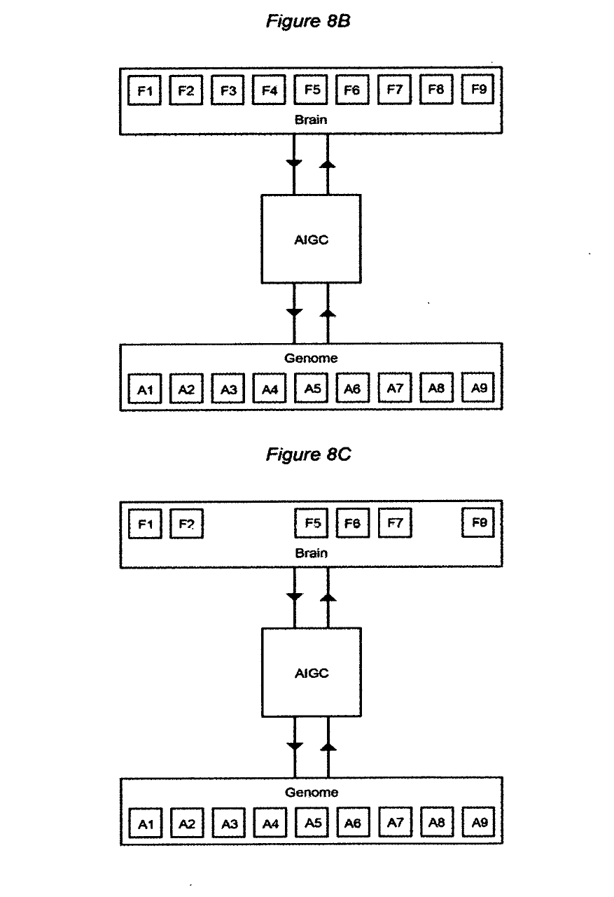

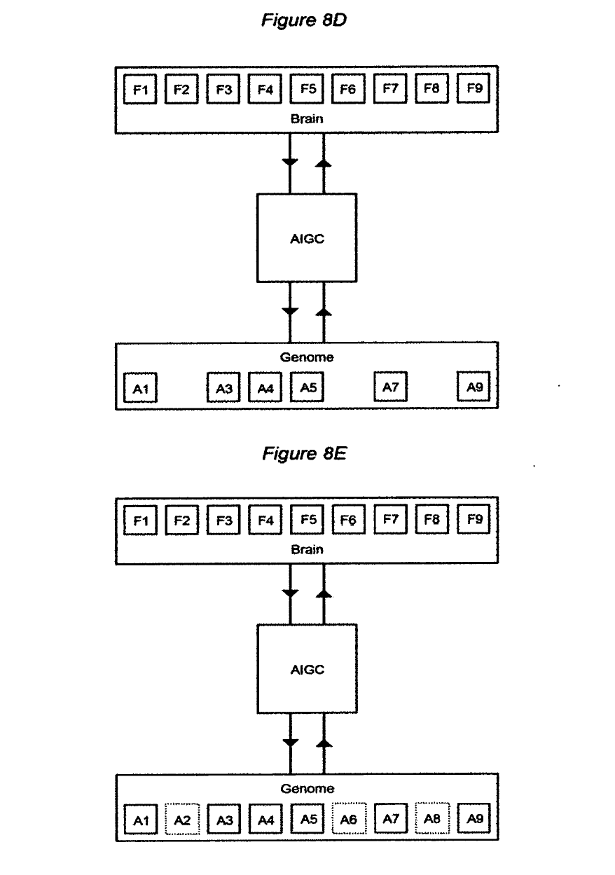

In some embodiments, a connection between the functions of the brain and the abilities of the genome is facilitated by an AIGC, as is shown in FIG. 8A where AIGC 801 connects brain 803 to genome 802. In order to split functions and abilities, code must be written and divided in a specific way, where the base code that enables the functionality is stored in the genome but the actual functions and/or function calls are stored in the brain. For example, in a machine designed to walk:

If the function itself is stored in the brain:
The genome contains the code that allows the brain to control the legs.
The brain contains the code that actually moves the legs.

If only the function call is stored in the brain:
The genome contains both the code that enables control and the function code to actually move the legs.
The brain contains function calls for named functions within the genome.

FIG. 8B shows these pairs as they are split between the genome and the brain, with each “F” number in the brain having a corresponding “A” number in the genome.

In some embodiments, a genome may contain abilities that do not have corresponding functions in the brain. This is shown in FIG. 8C. In such embodiments, the abilities, though present, cannot be used by the AI unless the functions are then implemented—either automatically through AI learning or through manual implementation.

In some embodiments, a genome may not contain abilities that correspond with functions within the brain of the AI, as shown in FIG. 8D. In such a situation, the functions would only be usable if the entirety of the code was present within the brain or accessible outside of the genome. In these embodiments, the unpaired functions of the brain may be made into abilities. By splitting the complete function code in one of the two ways explained above, part of the function can be made into an ability and transferred to the genome, as shown in FIG. 8E. This transfer may be done automatically, via the AIGC, or manually.

In some embodiments, the AIGC can use one genome to heal another genome that has any type of deficiency, as shown in FIGS. 4D to 4H. An example of this is shown in FIG. 11. When a genome is deficient—this may be detected through automatic or manual monitoring—the AIGC can find a copy of the genome without any deficiency or, at least, one that has the deficient parts of the deficient genome complete and without abnormality within itself, and copy the necessary parts from the normal genome to the deficient one.

In FIG. 11, genome 1102 is deficient. AIGC 1105 takes genome 1101—it having no deficiency—and copies the necessary data over to genome 1102, resulting in the now healed genome 1104. Since data has only been copied, genome 1101 remains unchanged, as is shown in its representation as genome 1103.

For genome healing and other genome-to-genome functions a network must exist between this. This network may be physical or non-physical; wired or wireless; and is facilitated by the AIGC. An example of this is shown in FIG. 10. Non-physical networks may see genomes in multiple directories of a system. Physical networks may see genomes stored on storage mediums and distributed around the physical body of a machine. In some embodiments, the AIGC is connected to each genome individually. In some embodiments, the AIGC is connected to genomes through other genomes—a genome chain. In some embodiments, both may be true, creating a genome web. In some embodiments, the AIGC may also be linked to the AI brain, even if the brain is its own singular connection and not connected to any genome.

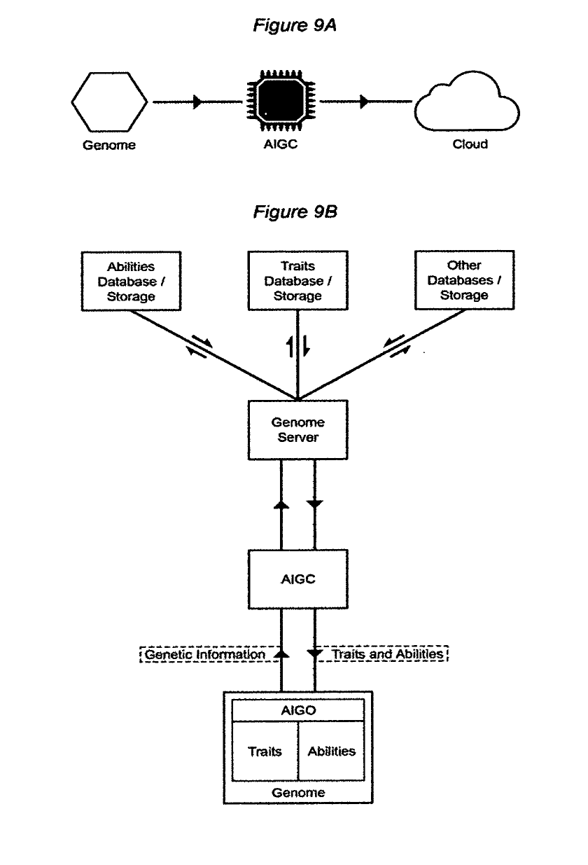

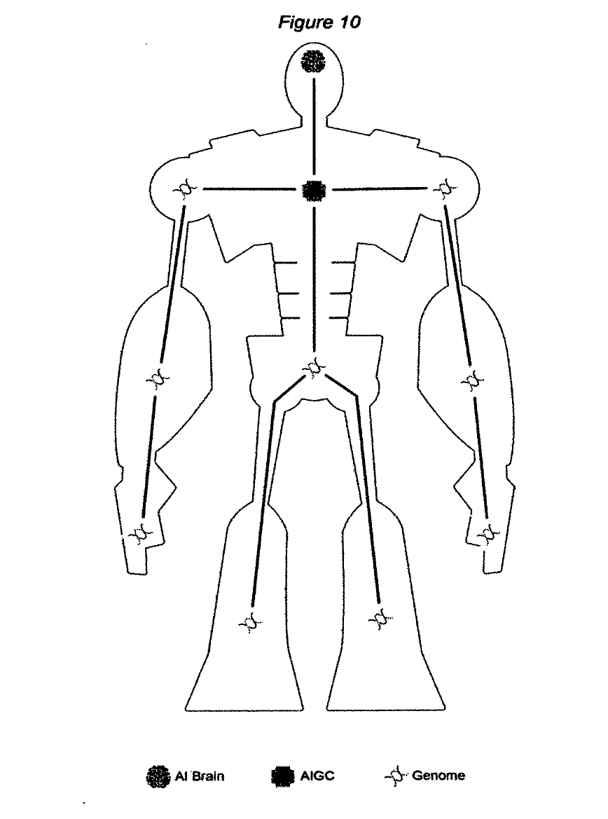

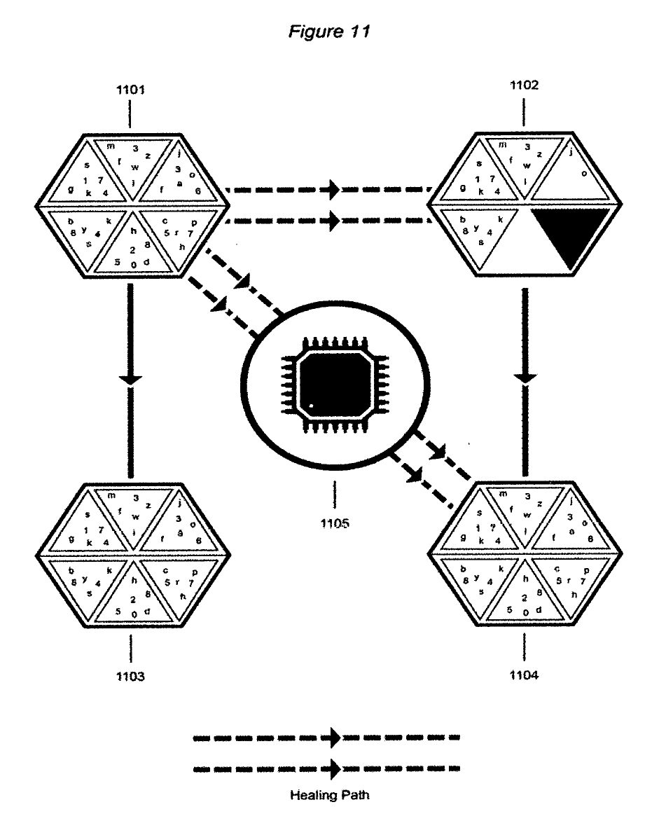

In some embodiments, a genome may be backed up to a server, such as a cloud. The AIGC only needs the abilities and permissions to copy and upload data. An example is shown in FIG. 9A, where the AIGC copies data from the genome to a cloud server.

In some embodiments, traits and/or abilities and/or other genetic features may be downloaded from a server based upon the genetic information contained within the AIGO. This is shown in FIG. 9B. The AIGC pulls genetic information from the AIGO of a genome and sends it to a server storing genetic features. The server processes the genetic information and retrieves the corresponding information from databases and files from storage before passing the information back to the AIGC, which implements it into the genome based on the AIGO specification.

In some embodiments, the information contained within the genome can be used to compile a system or machine. Reading the AIGO via the AIGC or directly, a system can use the genetic information, such as the traits and abilities for a model number, to put together a physical or non-physical system/machine that is compatible with the genome in use. In some embodiments, this process may also work in reverse, where system/machine information can be turned into genetic information based on the same required information (model number, ability/trait etc) and used to create a genome using a method similar to that shown in and accompanying FIGS. 8D and 8E. In these embodiments, the system/machine will need to:
Be designed so that the required data of said system/machine can be transferred to the AIGC of the genome and into the genome core itself in a compatible format; or
Deconstruct/reconfigure data—by itself or with the assistance of another system—before or during transfer to the AIGC and Into the genome core itself in a compatible format.

In some embodiments, values of an AI, such as those referred to and shown in patents GB1517146.5 and GB1409300.9 as ‘objects’ and ‘words/phrases’ respectively, may be inherited in a similar way to how traits and abilities are be inherited. However, there are additional requirements to do so, those being:
The AIGC needs to have a connection to where these values are stored in the AI (usually the brain) or the values data need to be made available to the AIGC, when necessary at least.
The AIGC needs to have abilities and permissions that allow it to access and copy the values data.
The AIGO needs to include the location of these values within the genome.

In some embodiments, the AIGO may contain the location of where values are stored outside of the genome.

In some embodiments, only some values are inherited. In some embodiments, all values are inherited.

In the event that values are only being inherited from a single genome, those values are copied/moved into the new genome. In the event that values are inherited from multiple genomes, handling methods need to be employed for duplicate values, such as but not limited to one or more of the following examples:
With values of different degrees/natures, the mean degree may be used. For example, if X from genome 1 is a degree of 6 and a 10 in genome 2, it is set at 8 in the inheriting genome.
For each duplicate value, one of the duplicates is randomly selected and implemented and the other discarded.
One is chosen that best suits the desired nature of the inheriting genome.

In some embodiments, the process of inheriting values may be allowed to include human intervention in order to help sort values. Human intervention may include any form of human involvement in the process, such as but not limited to one or more of the following:
A human manually sorting values;
A human and AI working together to decide the sorting of values; and
A human overseeing the automatic sorting of values.

In some embodiments, other qualities and features mentioned in the two referenced patents may be inherited, including any quality, feature or position of the Object, Value and Sensation System and/or the Productivity and Reaction System, such as sensitivity, productivity and reaction.

Inheritable qualities mentioned in the two referenced patents are, too, classed as traits.

The foregoing description, for purpose of explanation, has been described with reference to specific embodiments. However, the illustrative discussions above are not intended to be exhaustive or to limit the invention to the precise forms disclosed. Many modifications and variations are possible in view of the above teachings. The embodiments were chosen and described in order to best explain the principles of the invention and its practical applications, to thereby enable others skilled in the art to best utilize the invention and various embodiments with various modifications as are suited to the particular use contemplated.

1. An Artificial Intelligence Genome (AIG), wherein a modular, hierarchical structure of self-contained data within a system and/or machine contains and is used to give the AI in which it inhabits traits and/or abilities, without having direct control over the actions or operations of the AI, but while being able to influence, in part or in full, one or more of the traits, abilities, and/or functions of the AI.

2. The AIG of claim 1, wherein multiple genomes are present within a single system or machine.

3. The AIG of claim 2, wherein multiple genomes can be connected to create a network.

4. An Artificial Intelligence Genome Organiser (AIGO), wherein a list of data pertaining to the design and genetic information of an Artificial Intelligence Genome (AIG) contains one or more of the following, including but not limited to:
structural information;
identifying information;
ancestral information;
generational information;
traits and/or abilities;
trait/ability associated model numbers; and
trait/ability associated indications of inheritability.

5. The AIGO of claim 4, wherein it is used as a map of the internal structure of a genome, based on the locations and/or positions of what is contained within the genome that is listed within the AIGO.

6. The AIGO of claim 4, wherein the values for one or more sections of the AIGO are subject to randomisation, based on:
the different values used in the AIGOs of the genomes participating in an amalgamation process;
a pre-compiled list of possible values; and/or
a value an AIGC is able to determine is best.

7. An Artificial Intelligence Genome Controller (AIGC), wherein a program comprising one or more of the following:
abilities and permissions to create and/or handle an Artificial Intelligence Genome (AIG); and
abilities and permissions to create and/or handle an Artificial Intelligence Genome Organiser (AIGO);
facilitates and controls the automation of functions and tasks of or involving an AIG.

8. A computer implemented method, wherein an AI is able to evolve without human intervention through the use of an Artificial Intelligence Genome (AIG), an Artificial Intelligence Genome Organizer (AIGO), and an Artificial Intelligence Genome Controller (AIGC), the method comprising:
storing traits and/or abilities within an AIG;
storing genetic information about the genome within an AIGO; and
using an AIGC to control and manipulate an AIG based on the genetic information of an AIGO.

9. The computer implemented method of claim 8, wherein two or more genomes may undergo an amalgamation process which sees some or all of their genetic information and features combined to create one or more new genomes, with the one or more new genomes inheriting genetic traits and abilities.

10. The inheritance of claim 9, wherein traits and abilities can only be inherited when specific conditions are met that determine a trait/ability should be inherited, those conditions including but not limited to one or more of the following:
a frequency of use;
whether or not they have been superseded by a trait/ability that has been deemed superior; and
a factor scoring system.

11. The inheritance of claim 9, wherein one or more handling methods determine the positions of inherited duplicate values, the one or more handling methods including but not limited to:
calculating and using the mean degree of the same value;
randomly selecting which of the values to keep; and
choosing one that best suits the desired nature of the genome.

12. The handling methods of claim 11, wherein one or more methods of human intervention are used to help sort values, the one or more methods including but not limited to:
manually sorting values;
working together with an AI to decide the sorting of values; and
overseeing the automatic sorting of values.

13. The computer implemented method of claim 8, wherein abilities stored within a genome are part of a function-ability pairing.

14. The function-ability pairing of claim 13, wherein abilities can be created from the splitting of functions of the AI brain and transferred to the AIG via the AIGC.

15. The computer implemented method of claim 8, wherein the AIGC facilitates a connection to a server for one or more of the following purposes, including but not limited to:
the backing up of genome data; and
the downloading of genome data.

16. The computer implemented method of claim 8, wherein a deficient genome can be healed by copying healthy versions of the deficient parts from another genome into the deficient genome.

17. The computer implemented method of claim 8, wherein genetic information of an AIG is used to create systems and/or machines based on the identifying information of traits/abilities, such as model number and version number, to determine what parts the AIG is compatible with.

18. The computer implemented method of claim 8, wherein a method of creating a genome from a system or machine comprises:
the system/machine being designed so that the data that will be required as part of the genome can be transferred to the AIGC; and/or
the deconstructing/reconfiguring of data before or during transfer to the AIGC;
in a compatible format for the AIGC to be able to correctly build an AIGO and implement the data into the genome core.
